(19) United States
(12) Patent Application Publication
(10) Pub. No.: US 2018/0344520 A1
(43) Pub. Date: Dec. 6, 2018

(54) CONTACT LENS INSERTION ASSISTING ASSEMBLY

(71) Applicant: Mark Daniels, Edenton, NC (US)

(72) Inventor: Mark Daniels, Edenton, NC (US)

(21) Appl. No.: 15/611,069

(22) Filed: Jun. 1, 2017

(51) Int. Cl. A61F 9/00; A45C 11/00

(52) U.S. Cl. CPC A61F 9/0061; A45C 2011/007; A45C 11/005

(57) ABSTRACT

A contact lens insertion assisting assembly includes an applicator comprising a tube that has a top end and a bottom end. The top end is concavely shaped and this shape allows the top end to receive a contact lens. A central portion of the top end has a well therein extending downwardly toward the bottom end.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to contact lens application devices and more particularly pertains to a new contact lens application device for assisting a person in positioning a contact lens on their eye.

### BRIEF SUMMARY OF THE INVENTION

An embodiment of the disclosure meets the needs presented above by generally comprising an applicator comprising a tube that has a top end and a bottom end. The top end is concavely shaped and this shape allows the top end to receive a contact lens. A central portion of the top end has a well therein extending downwardly toward the bottom end.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a contact lens insertion assisting assembly according to an embodiment of the disclosure.

FIG. 2 is a perspective exploded view of an application of an embodiment of the disclosure.

FIG. 3 is a bottom view of an applicator of an embodiment of the disclosure.

FIG. 4 is a perspective view of a container of an embodiment of the disclosure.

FIG. 5 is a front view of a kite of an embodiment of the disclosure.

### DETAILED DESCRIPTION OF THE INVENTION

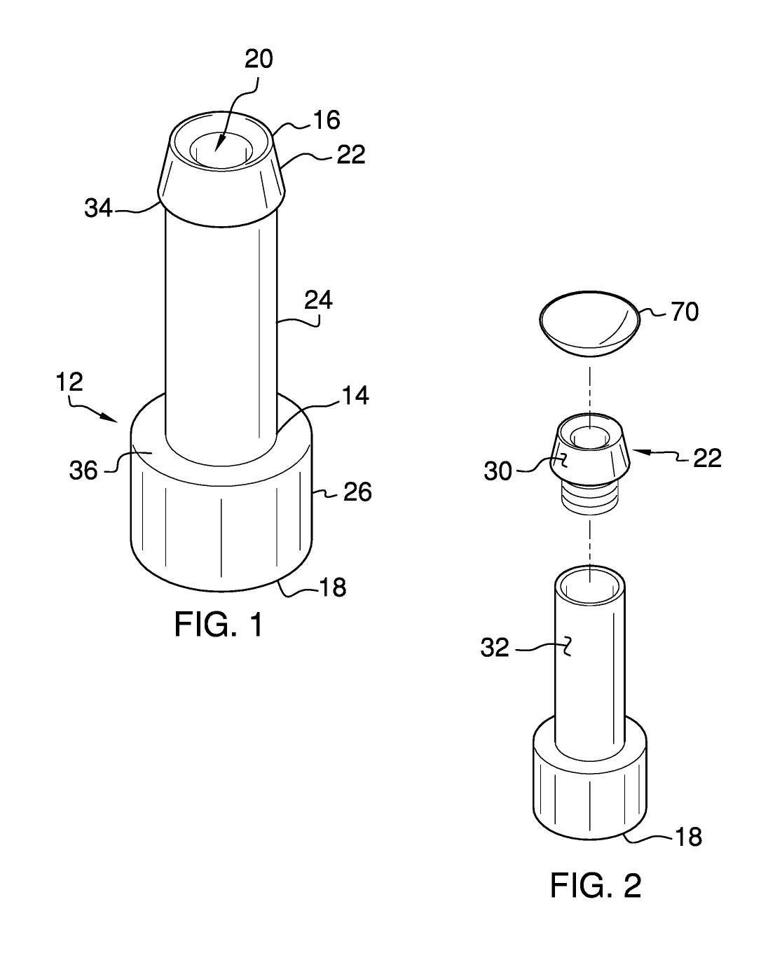

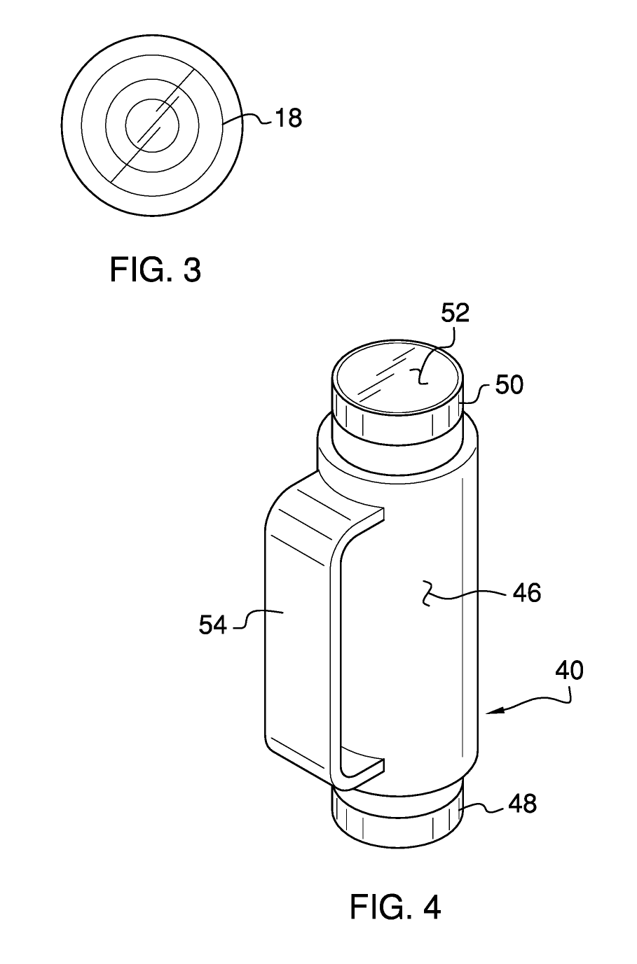

With reference now to the drawings, and in particular to FIGS. 1 through 5 thereof, a new contact lens application device embodying the principles and concepts of an embodiment of the disclosure and generally designated by the reference numeral 10 will be described.

As best illustrated in FIGS. 1 through 5, the contact lens insertion assisting assembly 10 generally comprises an applicator 12 including a tube 14 that has a top end 16 and a bottom end 18. The top end 16 is concavely shaped and is configured to receive a conventional contact lens 70. A central portion of the top end 16 has a well 20 therein extending downwardly toward the bottom end 16. The tube 14 includes an upper portion 22, a central portion 24 and a bottom portion 26 wherein the upper portion 22 includes the top end 16. At least one of the upper 22, central 24 or bottom 26 portions may comprise a translucent material to allow light to pass through the applicator 12 and into the well 20. Alternatively, the bottom end 18 may be clear or open and the well 20 may extend to the bottom end 18 to allow light to pass through the applicator 12 and outwardly of the top end 16. As a person inserts the contact lens 70 into their eye, this well 20 will become visible and thereby assure the person that they are properly positioning the contact lens 70 in their eye.

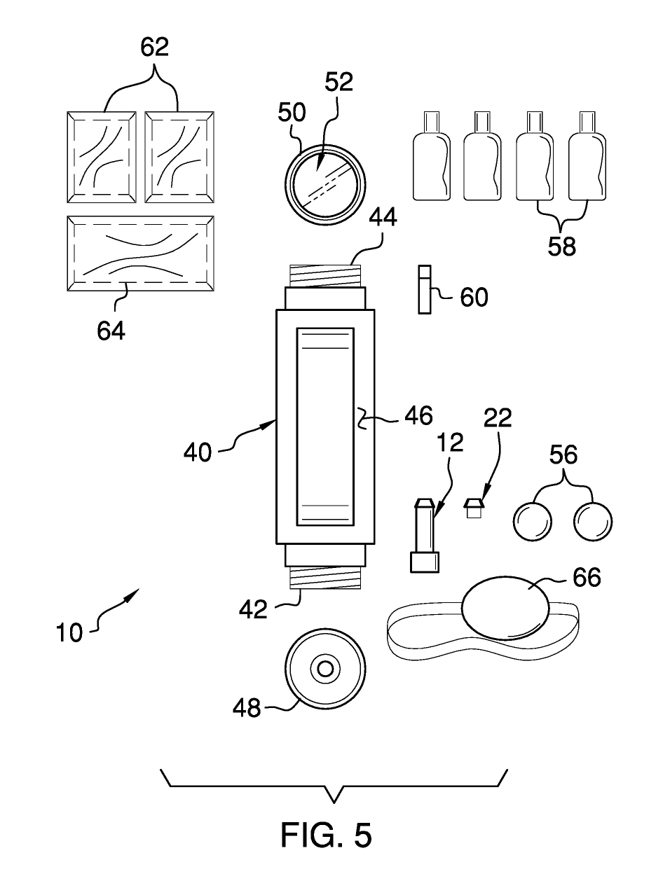

A peripheral surface 30 of the upper portion 22 may flare outwardly from the top end 16 to the central portion 24. The upper portion 22 may further comprise a resiliently flexible material such as an elastomer including rubbers, synthetic rubbers and silicon materials. If flared, the upper portion 22 extends outwardly from an outer surface 32 of the central portion 24 such that an upper shoulder 34 is defined between the upper 22 and central 24 portions. The upper portion 22 may be removably coupled to the central portion 24 either with threading or by friction fit. This will allow the upper portion 22 to be replaced for cleaning, as it wears or if its size is to be altered. Multiple ones of the upper portions 22 may be provided as shown in FIG. 5.

The bottom portion 26 has a greater diameter than the central portion 24 such that a lower shoulder 36 is defined between the central portion 24 and the lower portion 26. The lower 36 and upper 34 shoulders provide better gripping for the user of the applicator 12. The applicator 12 has a length from the bottom end 18 to the top end 16 generally between 2.5 inches and 4.0 inches and the central portion 24 has a diameter that is less than 0.75 inches.

A kit, as generally shown in FIG. 5, is provided that is configured to facilitate utilization of the applicator 12 and to protect it from being soiled. The kit comprises a container 40 that has a first end 42, a second end 44 and a perimeter wall 46 extending between the first 42 and second 44 ends. The first end 42 is open and removably receives the applicator 12 such that the applicator 12 is stored within the container 40. A first end cap 48 is removably positioned on the first end 42 to close the first end 42. The second end 44 is open and a second end cap 50 is removably positioned on the second end 44 to close the second end 44. The second end cap 50 has an outer end 52 comprising a mirror and the second end cap 50 may be comprised of a magnetized material. A loop 54 is attached to the perimeter wall 46 and is used to receive a belt for easy transportation of the container 40.

Within the container 40 multiple additional components may be provided that may be useful for the user of the applicator 12 and which are removably stored within the container 40. These additional components may include pair of contact lens cases 56, at least one bottle of contact solution 58, and least one contact lens remover 60 used for removing contact lenses from the contact cases 56. Additionally, one or more wipes 62 may be included, which are saturated with a cleaning solution, as well as at least one drying cloth 64. An eye patch 66 may also be positioned in the container 40.

In use, the assembly 10 is used for assisting a person who has difficulty inserting contact lenses 70 into their eyes. The user will place the contact lens 70 on the first end 16 of the applicator 12 and then apply lens solution on top of the contact lens 70. The user then bends over and moves their eye to the contract lens 70 until the contact lens 70 is moved onto the eye. The user will be able to see the well 20 in the first end 16 when the contact lens 70 is properly positioned on the eye.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A contact lens insertion assembly comprising:
an applicator comprising a tube having a top end and a bottom end, said top end being concavely shaped, said top end being configured to receive a contact lens, a central portion of said top end having a well therein extending downwardly toward said bottom end.

2. The contact lens insertion assembly according to claim 1, wherein said tube includes an upper portion, a central portion and a bottom portion wherein said upper portion includes said top end, a peripheral surface of said upper portion flaring outwardly from said top end to said central portion.

3. The contact lens insertion assembly according to claim 1, wherein said tube includes an upper portion, a central portion and a bottom portion wherein said upper portion includes said top end, said upper portion comprising a resiliently flexible material.

4. The contact lens insertion assembly according to claim 2, wherein said upper portion extends outwardly from an outer surface of said central portion such that an upper shoulder is defined between said upper and central portions.

5. The contact lens insertion assembly according to claim 4, wherein said bottom portion has a greater diameter than said central portion such that a lower shoulder is defined between said central portion and said lower portion.

6. The contact lens insertion assembly according to claim 2, wherein light passes through said applicator and outwardly of said well.

7. The contact lens insertion assembly according to claim 3, wherein light passes through said applicator and outwardly of said well.

8. The contact lens insertion assembly according to claim 2, wherein said upper portion being removably coupled to said central portion.

9. The contact lens insertion assembly according to claim 3, wherein said upper portion being removably coupled to said central portion.

10. The contact lens insertion assembly according to claim 2, wherein said applicator having a length from said bottom end to said top end between 2.5 inches and 4.0 inches, said central portion having a diameter being less than 0.75 inches.

11. The contact lens insertion assembly according to claim 3, wherein said applicator has a length from said bottom end to said top end between 2.5 inches and 4.0 inches, said central portion having a diameter being less than 0.75 inches.

12. The contact lens insertion assembly according to claim 1, further including:
a kit configured to facilitate utilization of said applicator, said kit comprising:
a container having a first end, a second end and a perimeter wall extending between said first and second ends, said first end being open and removably receiving said applicator such that said applicator is stored within said container, a first end cap being removably positioned on said first end to close said first end.

13. The contact lens insertion assembly according to claim 12, wherein said second end being open and a second end cap being removably positioned on said second end to close said second end.

14. The contact lens insertion assembly according to claim 13, wherein said second end cap has an outer end comprising a mirror.

15. The contact lens insertion assembly according to claim 14, wherein said second end cap is comprised of a magnetized material.

16. The contact lens insertion assembly according to claim 12, further including a loop being attached to said perimeter wall, said loop being configured to receive a belt.

17. The contact lens insertion assembly according to claim 12, further including:
a pair of contact lens cases being removably positioned in said container;
at least one bottle of contact solution being removably positioned in said container.

18. The contact lens insertion assembly according to claim 17, further including:
at least one contact lens remover being positioned in said container;
at least one wipe being positioned in said container;
at least one drying cloth being positioned in said container; and
at least one eye patch being positioned in said container.

19. A contact lens insertion assembly comprising:
an applicator comprising a tube having a top end and a bottom end, said top end being concavely shaped, said top end being configured to receive a contact lens, a central portion of said top end having a well therein extending downwardly toward said bottom end, said tube including an upper portion, a central portion and a bottom portion wherein said upper portion includes said top end, a peripheral surface of said upper portion flaring outwardly from said top end to said central portion, said upper portion comprising a resiliently flexible material, said upper portion extending outwardly from an outer surface of said central portion such that an upper shoulder is defined between said upper and central portions, said bottom portion having a greater diameter than said central portion such that a lower shoulder is defined between said central portion and said lower portion, wherein light passes through said applicator and outwardly of said well, said upper portion being removably coupled to said central portion, said applicator having a length from said bottom end to said top end between 2.5 inches and 4.0 inches, said central portion having a diameter being less than 0.75 inches.

20. A contact lens insertion assembly comprising:
an applicator comprising a tube having a top end and a bottom end, said top end being concavely shaped, said top end being configured to receive a contact lens, a central portion of said top end having a well therein extending downwardly toward said bottom end, said tube including an upper portion, a central portion and a bottom portion wherein said upper portion includes said top end, a peripheral surface of said upper portion flaring outwardly from said top end to said central portion, said upper portion comprising a resiliently flexible material, said upper portion extending outwardly from an outer surface of said central portion such that an upper shoulder is defined between said upper and central portions, said bottom portion having a greater diameter than said central portion such that a lower shoulder is defined between said central portion and said lower portion, wherein light passes through said applicator and outwardly of said well, said upper portion being removably coupled to said central portion, said applicator having a length from said bottom end to said top end between 2.5 inches and 4.0 inches, said central portion having a diameter being less than 0.75 inches;
a kit configured to facilitate utilization of said applicator, said kit comprising:
a container having a first end, a second end and a perimeter wall extending between said first and second ends, said first end being open and removably receiving said applicator such that said applicator is stored within said container, a first end cap being removably positioned on said first end to close said first end, said second end being open and a second end cap being removably positioned on said second end to close said second end, said second end cap having an outer end comprising a mirror, said second end cap being comprised of a magnetized material, a loop being attached to said perimeter wall, said loop being configured to receive a belt;
a pair of contact lens cases being removably positioned in said container;
at least one bottle of contact solution being removably positioned in said container;
at least one contact lens remover being positioned in said container;
at least one wipe being positioned in said container;
at least one drying cloth being positioned in said container; and
at least one eye patch being positioned in said container.
